(19) United States
(12) Patent Application Publication
(10) Pub. No.: US 2011/0318436 A1
(43) Pub. Date: Dec. 29, 2011

(54) HERBAL SOLID FORMULATION AND PROCESS FOR PREPARING THE SAME

(75) Inventors: Shankar Kumar MITRA, Bangalore (IN); Uddagiri Venkanna BABU, Bangalore (IN); Ekta SAXENA, Bangalore (IN); Anu Vrat SHARMA, Bangalore (IN)

(73) Assignee: HIMALAYA GLOBAL HOLDINGS LTD, Grand Cayman (KY)

(21) Appl. No.: 13/011,724

(22) Filed: Jan. 21, 2011

Related U.S. Application Data
(63) Continuation-in-part of application No. 13/003,543.

(51) Int. Cl. A61K 36/8962; A61P 3/02

(52) U.S. Cl. 424/754

(57) ABSTRACT

Disclosed is a herbal solid formulation of Allium sativum extracts essentially free of excipients and preservatives and process for preparing the same.

This application is a continuation-in-part of U.S. application Ser. No. 13/003,543 which is a U.S. national stage entry of PCT application number PCT/IB2008/001797 filed Jul. 9, 2008 the entire disclosures of which are expressly incorporated by reference herein.

### FIELD OF THE INVENTION

This invention, in general relates to an herbal solid formulation. In particular, the present invention provides a herbal solid formulation of Allium sativum extracts without using any excipients and preservatives and process for preparing the same.

### BACKGROUND OF THE INVENTION

Herbal supplements have been witnessing tremendous growth and acceptance among the consumers during the last decade due to their safety and efficacy. Unlike allopathic medications, herbal extracts are safe and devoid of any side effects. There is a growing concern among the consumers worldwide using naturally derived products and avoiding synthetic chemicals in their food, personal care products and daily health supplements. Many herbal products those are available in the market as tablets and capsule using synthetic excipients such as binders, lubricants and diluents and preservatives such as Parabens and salts of Benzoic acids etc. These excipients and preservatives are reported to have toxic and side affects from time to time.

Pharmaceutical dosage forms such as tablets and capsules should have certain properties such as hardness, friability, disintegration time (DT), stability and delivery of the drug to give required therapeutic benefits to the patient. These properties are achieved using the excipients such as binders, lubricants and diluents.

In the recent times there is a growing concern among consumers regarding the presence of pesticides and heavy metals in natural herbal materials. It is very important to cultivate and collect the herbal materials that are free from pesticides residues. The United States Department of Agriculture (USDA) has framed certain approved practices and procedures for organic farming under National Organic Programme. The herbal materials cultivated and collected under NOP certification would be free from pesticides and low heavy metals content as per WHO and USFDA guidelines.

It is therefore very important and challenging task to develop a process of manufacturing herbal tablets using organic herbal extract and plant powder without using any synthetic extraction solvents, excipient and preservative and yet to meet all pharmaceutical standards as dietary supplements, nutraceutical or herbal medicinal preparation.

### RELATED ART

U.S. Pat. No. 6,207,189 by Mercati et al disclose a process for the production of tablets and capsules of natural substances of vegetable origin wherein dry extracts and micronized powders of one or more medicinal herbs in appropriate proportions are blended and subjected to steam pressure followed by drying, preparation of granules and compression to tablets.

U.S. Pat. No. 6,468,563 by Schmidt et al. discloses a process for producing rapidly disintegrating pharmaceutical formulation containing an extract and lubricant and compressing the blend to form the pharmaceutical formulation.

U.S. Pat. No. 5,705,152 by Plummer discloses an antimicrobial composition comprises antimicrobial material derived from the plant family Allium and non-pathogenic microorganisms of at least one species. The preferred source of the plant extract is Allium sativum (garlic). The formulation may be provided in any suitable form, such as a powder, tablet, capsule or similar form, or an aqueous solution containing the formulation as an active ingredient.

U.S. Pat. No. 6,270,803 by Blatt et al. discloses orally-administrable formulation for the controlled release of granulated garlic. The formulation comprises particles of granulated garlic coated with a film comprising a mixture of at least one water soluble polymer and at least one water insoluble polymer. The formulation is in a form of a matrix tablet, a multicomponent formulation, a microcapsule of generally spherical shape, a microcapsule of generally non-spherical shape, a capsule containing microcapsules, or a tablet containing microcapsules U.S. Pat. No. 4,849,218 by Hess et al. discloses an oral garlic preparation containing garlic powder as well as the enzyme allinase, in which each of the components is spatially separated from the other and are recombined only after having been orally administered in the form of tablet or capsule and have storage stability.

### SUMMARY OF THE INVENTION

It is a principal object of the invention to provide a herbal solid formulation of Allium sativum extracts essentially free of additives/excipients and preservatives and providing required quantity of active constituents per dose.

It is another object of the invention to provide an herbal solid formulation of herb Allium sativum extracts having reduced side effects and toxicity.

It is yet another object of the invention to provide a herbal solid formulation of herb Allium sativum extracts essentially free of excipients/additives and preservatives and having desired friability, disintegration time and hardness.

It is still another object of the invention to provide a method for preparing extract of herb Allium sativum used to prepare the solid formulation.

The above and other objects of the present invention are attained according to following preferred embodiments of the present invention. However the scope of the invention is not restricted to the particular embodiments discussed herein after.

In accordance with one preferred embodiment of the present invention, there is provided a herbal solid formulation comprises a blend of freeze dried powder of herb Allium sativum and water extract of herb Allium sativum along with powder of Allium sativum, wherein said blend of extract and said powder of herb is mixed in a ratio of about 1:0.5 to about 1:10.

In accordance with one preferred embodiment of the present invention, there is provided a herbal solid formulation comprises a blend of freeze dried powder of herb Allium sativum and water extract of herb Allium sativum along with powder of Allium sativum, wherein said herbal solid formulation is essentially free of additives/excipients.

In accordance with another preferred embodiment of the invention there is provided a process for preparing a herbal solid formulation essentially free of additives/excipients comprising granulating a blend of freeze dried powder and water extract of Allium sativum, and lubricating the granulated blend by adding the powder of Allium sativum and preparing the solid formulation.

In accordance with yet another preferred embodiment of the invention, the powder of herb is obtained by pulverizing the herb to a powder having mesh size preferably between about 20 to about 100, more preferably between 20 to 80.

In accordance with yet another embodiment of the present invention, the extract of Allium sativum is passed through a mesh having size between about 20 to 80.

In accordance with still another embodiment of the present invention, wherein the granulation of the blend of extracts and powder of the herb is carried out in presence of a solvent, preferably alcohol, water and grain alcohol or combination thereof.

In accordance with yet another embodiment of the present invention, there is provided a process for preparing the extract of the herb by percolation, hot soxhalation or refluxing followed by filtration and concentration to dryness at optimum temperature.

### BRIEF DESCRIPTION OF DRAWINGS

Further objects of the present invention together with additional features contributing thereto and advantages accruing there from will be apparent from the following description of preferred embodiments of the invention which are shown in the accompanying drawing FIGURES, wherein:

FIG. 1 illustrates HPLC chromatogram of garlic

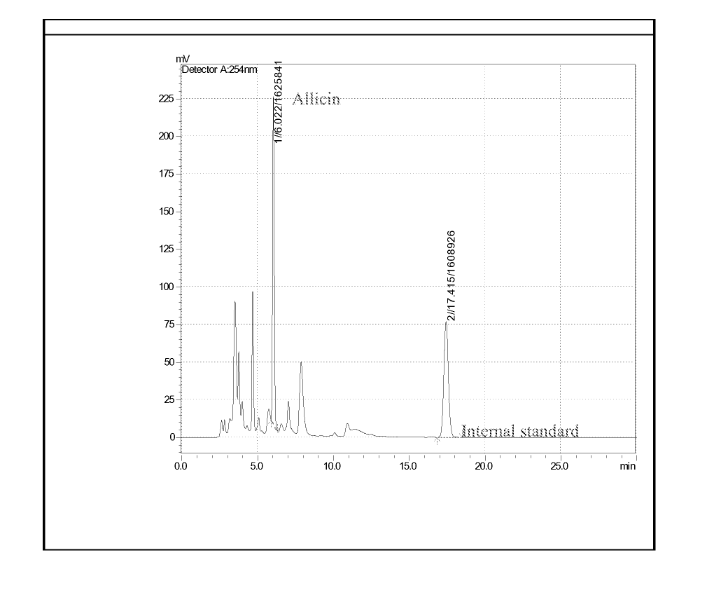

### DETAILED DESCRIPTION OF THE INVENTION

While this specification concludes with claims particularly pointing out and distinctly claiming that, which is regarded as the invention, it is anticipated that the invention can be more readily understood through reading the following detailed description of the invention and study of the included examples.

The present invention provides an herbal solid formulation essentially free of excipients/additives or preservatives, wherein said formulation comprises a blend of freeze dried garlic powder, water extract and aerial parts powder of Allium Sativum.

The present invention provides an herbal solid formulation essentially free of excipients/additives or preservatives, wherein said formulation comprises a blend of freeze dried powder and water extract of Allium sativum along with powder Allium sativum and a process for preparing the same.

The bulbs of Allium sativum were depoded, cleaned with water and chopped to reduce the size. The chopped material is freeze dried, hand crushed and passed though the mesh having size preferably between #10 to 100.

The extracts of the herb is prepared by employing percolation, hot soxhalation or refluxing method using a solvent, followed by filtration and concentration on a rotatory evaporator on steam bath at optimum temperature and under reduced pressure. The solvent employed includes alcohol, organic grain alcohol, ethanol or water or combinations thereof, preferably grain alcohol. The extract is dried and passes through a mesh having size preferably between #20 to 80.

The powder of the herb is prepared by pulverizing the aerial parts of herb to a powder of different mesh sizes based on the requirement, preferably between about 20 to about 100.

The freeze dried powder, extract and the powder of the herb is mixed in a predetermined ratio preferably between about 1:0.5 to about 1:10 for optimum granulation to prepare the herbal solid formulation. The granulation is performed using a solvent system followed by passing the granules through a mesh having size preferably between 12 to 24#. The solvent system employed for granulating the mixture includes grain alcohol, water or combination thereof.

All extracts, granules and tablets are subjected to standardization by High Performance Thin Layer Chromatography (HPTLC) and High Performance Liquid Chromatography (HPLC) for identification and quantitative estimation of active marker compounds. The extracts were evaluated for toxicity studies in rats and tablets for safety studies in healthy human volunteers.

The solid formulation according to the present invention has desired hardness preferably between about 2 to 8 kg/cm², preferably 3 to about 4 kg/cm², friability less than about 1% w/w and disintegration time less than about 60 min, preferably between 5-30 min. The solid formulation complies with USFDA guidelines.

According to the present invention, the disclosed solid formulation is preferably granules, tablet or capsule.

The following non-limiting examples illustrate specific embodiments of the present invention. They are, not intended to be limiting the scope of present invention in any way.

### Example-1

### Preparation of Allium sativum (Garlic) Water Extract

Approximately 100 Kg of shade dried Organic garlic bulbs were coarsely powdered and subjected to extraction with 400 Liters of purified water by percolation method at room temperature for 24 hours. The water extractions thus obtained were filtered through muslin cloth and concentrated to thick paste. After achieving the desired total solid content, the soft extract was spray dried to a free flowing dry extract powder. The dried extract was passed through #40 mesh stainless steel sieves

### Example-2

### Preparation of Allium sativum (Garlic) Powder by Freeze Drying

The fresh bulbs of Organic Garlic were depoded with its outer skin and subjected to cleaning with fresh water. The cleaned material was chopped randomly in mechanized cutter. The material was transferred into stainless steel trays in freeze dryer. The material is frozen to −25° C. for 4-5 hrs. The frozen material was loaded in to the chamber for drying under vacuum at 0.65 to 0.80 mBar and temperature from −25° C. to +30° C. The dried material was unloaded and checked for moisture content. The dried material was subjected to hand crushing and passed through #16 mesh size stainless steel sieves.

### Example-3

### Preparation of Allium sativum (Garlic) Aerial Parts Powder by Pulverization

The fresh aerial parts material of Organic Allium sativum (Garlic) was cleaned with purified water and shade dried. The shade dried materials was chopped into small pieces and then pulverized to coarse powder. The coarse powder was again pulverized and passed through #80 mesh stainless steel sieves to obtain fine garlic aerial parts powder.

### Example-4

### Preparation of Organic Garlic Granules (Formula-1)

TABLE 1

| | | Weight per | Weight per |
|---|---|---|---|
| S. No. | Name of the Material | Tablet in mg | Batch In kg |
| 1 | Allium sativum (water | 170.00 | 17.00 |
| | extract) (#40 mesh) | | |
| 2 | Allium sativum aerial | 150.00 | 15.00 |
| | parts (# 80mesh) | | |

### Example-5

### Preparation of Organic Garlic Granules Lubrication (Formula-2)

TABLE 2

| | | Weight per | Weight per |
|---|---|---|---|
| S. No. | Name of material | Tablet in mg | Batch in Kg |
| 1 | Allium sativum freeze dried | 320.00 | 32.00 |
| | material (#16 mesh) | | |
| 2 | Allium sativum granules (#20 | 325.00 | 32.50 |
| | mesh) | | |
| 3 | Allium sativum aerial parts | 55.00 | 5.50 |
| | (# 80 mesh) | | |
| | Total | 700.00 | 70.00 |

### Example-6

### Procedure for Sterilization of Organic Garlic Aerial Parts

1. Transfer weighed quantity of Allium sativum aerial parts in trays and sterilize the materials @ 160° C. for 2 hour
2. Unload the sterilized materials in to double-lined poly bags separately and keep in air tight containers.
3. Samples were analysed for LOD, BD and microbial analysis as per Table-3

TABLE 3

| | Parameter | Standard values |
|---|---|---|
| 1 | LOD | 1.0-4.0% |
| 2 | BD | 0.25-0.5 g/ml |
| 3 | TVAC | NMT 5000 cfu/gm |
| 4 | Fungal count | NMT 10 cfu/gm |

### Example-7

### Procedure for Preparation of Organic Garlic Granules

1. Transfer about 15.0 kg of Purified water into a clean Stainless Steel Vessel for preparation of granulation fluid
2. Charged 17.0 Kg of Allium sativum water extract, 15.0 kg of Allium sativum aerial parts into the RMG, mix for 5 minute for granulation
3. Slowly add the granulation fluid to the RMG containing the Sifted Allium sativum water extract, Allium sativum aerial parts.
4. Top the mixer and scrape off the mass from the sides and bottom.
5. Continue mixing by operating the impeller at high speed with Chopper ON for about 3 minutes.
6. Add additional quantity of purified water, if required.
7. Discharge the mass from the RMG.
8. Mill the Wet Mass obtained in Multi mill fitted with 8 mm screen.
9. Dry the Wet mass obtained in tray Drier at about 70° C. to 80° C. for about 60 minutes.
10. Sift the dried granules using a Sifter fitted with sieve #16.
11 Analysis of granules as per the specification mentioned in Table-4

TABLE 4

| | Parameter | Standard value |
|---|---|---|
| 1 | LOD | 2.0-6.0% |
| 2 | BD | 0.35-0.70 g/ml |
| 4 | TVAC | NMT 5000 cfu/gm |
| 5 | Fungal count | NMT 10 cfu/gm |

### Example-8

### Procedure for Lubrication and Compression of Organic Garlic Granules

1. Transfer the sifted Allium sativum granules into the blending area
2. Transfer the sifted Allium sativum aerial parts lubricant
3. Transfer the sifted Allium sativum freeze dried material (#16-20 mesh)
4. Load 5.5 kg of Allium sativum aerial parts lubricant, 32.50 Kg of the # 20 mesh sized garlic granules and 32 Kg of Organic Freeze dried garlic material into the double cone blender
5. Blend the ingredients for about 6 minutes at 10-11 RPM.
6. Check the Temperature and Relative humidity of the area and record for compression of tablets
7. Transfer the granulated blend material into the compression area. Punch size—17×8 mm caplet, upper and lower punch plain.
8. The punched caplets would comply the following finished product specifications.

### Example-9

### Finished Product Specifications of Organic Garlic Caplets

1. Theoretical average weight: 700 mg
2. Weight uniformity: 700 mg±5% (665 mg to 735 mg)
3. Weight of 20 tablets: 14.00 g±3% (13.58 gm to 14.42 gm)
4. Tablet thickness: 3.5 to 6.5 mm
5. Tablet hardness: 2.0 to 8.0 Kg/cm²
6. Friability: NMT 1.0% W/W
7. Disintegration time: NMT 30 min
8. Allicin (By HPLC): NLT 1600 μg/Caplet

### Example-10

### Analytical Procedure for Estimation of Allicin in Garlic Cloves/Freeze Dried Materials/Caplets

Introduction: The method is carried out as per BP using butyl parahydroxybenzoate R as the internal standard. Carry out the assay as per timelines mentioned in the procedure.
Internal standard solution (0.02 g): Weigh accurately 20 mg (0.02 g) of butyl parahydroxybenzoate R in 100 ml volumetric flask, dissolve with methanol:water (1:1) mixture by sonication and make the volume up to the mark with methanol:water (1:1) mixture.
Sample preparation: Weigh accurately 0.8 g of the powdered garlic powder into 100 ml beaker and add 20 ml of water and homogenize the mixture in an ultrasonic bath at 4° C. (using ice cubes) for 5 minutes (use timer). Allow to stand to room temperature for 30 minutes (use timer). Transfer homogenized mixture in 20 ml centrifuge tube and centrifuge for 30 minutes (use timer) at 2500 rpm.
Preparation of dilution mixture: 1% v/v solution of anhydrous formic acid:methanol (40:60).
Stock solution: Dilute 10 ml of the supernatant to 25 ml with a dilution mixture.
Keep the sample in refrigerator. Remove it before 10 minutes of injection. Transfer the stock solution in 20 ml centrifuge tube and centrifuge for 5 minutes (use timer) at 2500 rpm. Place 0.5 ml of the internal standard solution in a 10 ml volumetric flask and make up the volume up to the mark with supernatant (centrifuged sample).

### Chromatographic Conditions:

Column: C₁₈ stainless steel column with 0.25 m long and 4 mm in internal diameter packed with silanised octadecylsilyl silica gel (5 μm) for chromatography
Wavelength: 254 nm
Flow rate: 0.8 ml/min
Volume of injection: 20 μl
Mobile phase: 1% v/v solution of Anhydrous formic acid:Methanol (40:60).
Run time: 30 minutes
   Procedure: Inject 20 μl of the internal standard solution and record the chromatogram to identify internal standard peak. Generally the internal standard peak is obtained between 14 and 18 minutes.
   Inject 20 μl of the sample solution and record the chromatogram. The peak corresponding to relative retention time of 0.33 to 0.38 with respect to internal standard in same chromatogram is considered as an allicin peak (the peak is observed approximately about 5-7 minutes as shown in chromatogram). Calculate the content of allicin as per the following formula.

### Calculation:

$$\% of allicin = \frac{{S}_{1} \times {m}_{2} \times 22.75}{{S}_{2} \times {m}_{1}}$$

S₁=Area of the peak corresponding to allicin (RRT 0.33 to 0.38).
S₂=Area of the peak corresponding to butyl parahydroxybenzoate peak obtained with sample solution in same chromatogram.
m₁=Mass of the sample taken in grams.
m₂=Mass of butyl parahydroxybenzoate in grams in 100 ml of the internal standard solution.

### Example-9

### Estimation of Amino Acids of Garlic Water Extract by Amino Acid Analyzer

TABLE 9

| S. No. | Name of the Amino acid | Qty of A. acid (%) |
|---|---|---|
| 1 | Asparagine | 1.64 |
| 2 | Glutamine | 4.18 |
| 3 | Serine | 0.54 |
| 4 | Hydroxyproline | 0.073 |
| 5 | Glycine | 0.6255 |
| 6 | Histidine | 0.199 |
| 7 | Argenine | 1.067 |
| 8 | Threonine | 0.43 |
| 9 | Alanine | 1.065 |
| 10 | Proline | 0.426 |
| 11 | Tyrosine | 0.280 |
| 12 | Valine | 0.634 |
| 13 | Methionine | 0.324 |
| 14 | Cysteic acid | 1.027 |
| 15 | Isoleucine | 0.33 |
| 16 | Leucine | 0.76 |
| 17 | Phenylalanine | 0.47 |
| 18 | Lysine | 0.3075 |

### CONCLUSION

It is evident from the above examples that the preparation of herbal solid formulation having the required content of Allicin and other Pharmaceutical properties of tablets is obtained by specialized process comprising the granulation of garlic water extract and autoclaved aerial parts powder and then the granulated mixture was lubricated with freeze dried garlic and autoclaved plant powder to compress into tablets. By this unique process, we can avoid the degradation of Allicin during granulation and compression procedure and also could be able to achieve the required parameters of disintegration, friability and hardness etc. The final tablets obtained by this procedure contain the active content Allicin at not less than 1600 micro gram per tablet along with blend of 16 aminoacids.

While this invention has been described in detail with reference to certain preferred embodiments, it should be appreciated that the present invention is not limited to those precise embodiments. Rather, in view of the present disclosure, which describes the current best mode for practicing the invention, many modifications and variations would present themselves to those skilled in the art without departing from the scope and spirit of this invention.

We claim:

1. A herbal solid formulation comprising a blend of freeze dried powder of Allium sativum and water extract of Allium sativum along with autoclaved powder of Allium sativum, wherein said blend of extract and said powder of herb is mixed in a ratio of about 1:0.5 to about 1:10.

2. The herbal solid formulation of claim 1, wherein said herbal solid formulation is essentially free of additives/excipients.

3. The process of claim 1, wherein the extract/powder of the Allium sativum is obtained using bulb & aerial.

4. The herbal solid formulation of claim 1, wherein said solid formulation is preferably granules, tablet or capsule.

5. The herbal solid formulation of claim 1, wherein said solid formulation is a tablet or caplet.

6. The herbal solid formulation of claim 5, wherein the tablet is having hardness of about 2 to about 8 kg/cm², a friability of less than about 1% w/w and disintegration time is less than about 60 min.

7. The herbal solid formulation of claim 6, wherein the tablet is having disintegration time is less than about 10 min.

8. The herbal solid formulation of claim 1, wherein the tablet is having Allicin content not less than 1600 microgram per tablet.

9. A herbal solid formulation of claim 1, wherein the tablet is having mixture of amino acids comprising Asparagine, Glutamine, Serine, Hydroxyproline, Glycine, Histidine, Argenine, Threonine, Alanine, Proline, Tyrosine, Valine, Methionine, Cysteic acid, Isoleucine, Leucine, Phenylalanine and Lysine.

10. A process for preparing a herbal solid formulation as claimed in claim 1, comprising:
granulating a blend of water extract and autoclaved plant powder from aerial parts;
lubricating the granulated mixture by adding the powder of Allium sativum and freeze dried garlic powder containing the required amount of Allicin; and
preparing the solid formulation.

11. The process of claim 10, wherein the extract of Allium sativum is obtained by employing percolation, hot soxhalation or refluxing.

12. The process of claim 9, wherein the percolation, hot soxhalation or refluxing method is performed in the presence of a solvent selected from alcohol, water, grain alcohol or combinations thereof and preferably water.

13. The process of claim 10, wherein the water extract of Allium sativum is passed through a mesh having size between about 20 to 80.

14. The process of claim 10, wherein the powder of Allium sativum is obtained by pulversing the herb to a powder having mesh size between about 20 to about 100.

15. The process of claim 10, wherein the granulation is carried out by employing a solvent system selected from water, grain alcohol or combinations thereof and preferably water.

16. The process of claim 10, wherein the granules are passed through a mesh having size between about 12 to 24.
